(19) United States
(12) Patent Application Publication
(10) Pub. No.: US 2004/0234612 A1
(43) Pub. Date: Nov. 25, 2004

(54) Cosmetic composition comprising at least one polymer particle dispersed in at least one liquid fatty phase and at least one compound plasticizing the polymer

(76) Inventors: Xavier Blin, Paris (FR); Caroline Lebre, Thiais (FR); Bertrand Lion, Luzarches (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/785,093

(22) Filed: Feb. 25, 2004

Related U.S. Application Data
(60) Provisional application No. 60/452,064, filed on Mar. 6, 2003.

(30) Foreign Application Priority Data
Feb. 25, 2003 (FR) 03 50035

(51) Int. Cl. A61K 9/14

(52) U.S. Cl. 424/489

(57) ABSTRACT

The present disclosure relates to a composition, for example, a cosmetic composition for caring for or making up the skin and/or epidermal derivatives and/or lips, which comprises a) at least one liquid fatty phase, b) a dispersion of at least one polymer particle dispersed in the at least one liquid fatty phase, and c) at least one compound chosen from esters of aliphatic and aromatic polycarboxylic acids and aliphatic and aromatic alcohols comprising from 1 to 10 carbon atoms, the at least one compound being present in an amount sufficient to plasticize the at least one polymer particle. The present invention likewise relates to a cosmetic composition for caring for and making up the skin and/or epidermal derivatives and/or lips, which comprises a physiologically acceptable medium comprising a) at least one liquid fatty phase, b) a dispersion of at least one polymer particle dispersed in the at least one liquid fatty phase, and c) at least one compound having a solubility parameter, δₕ, ranging from 5.5 to 11 (J/cm³)^{1/2}, the compound being present in an amount sufficient to plasticize the at least one polymer particle.

This application claims benefit of U.S. Provisional Application No. 60/452,064, filed Mar. 10, 2003.

Disclosed herein is a composition for use, for example, in the cosmetics field, comprising

a) at least one liquid fatty phase,

b) a dispersion of at least one polymer particle dispersed in the at least one liquid fatty phase, and

c) at least one compound chosen from esters of aliphatic and aromatic polycarboxylic acids and aliphatic and aromatic alcohols comprising from 1 to 10 carbon atoms, the at least one compound being present in an amount sufficient to plasticize the at least one polymer particle.

The composition disclosed herein may be used, for example, for caring for or making up keratin materials, such as the skin both of the face and of the human body, including the scalp, the epidermal derivatives such as the eyelashes, eyebrows, nails and hair, and also the lips and upper or lower eyelids of human beings.

In one embodiment, the composition disclosed herein provides access to makeup with staying power which does not undergo transfer and/or is easy to apply.

Non-transfer or no-transfer compositions are compositions which on a substrate form a deposit which does not undergo deposition at least in part onto articles (glasses, clothes, cigarettes) with which the compositions are brought into contact.

A makeup with staying power is one whose colour and/or gloss lasts throughout the day and which is resistant to trials, in particular to eating in the case of a lipstick.

A composition is easy to apply, in the sense that it goes on smoothly and spreads readily, and/or that the mass thereof deposited in one pass is sufficient to give a satisfactory makeup result.

One aspect of the present disclosure relates to a cosmetic product comprising at least two compositions which are applied in succession, in particular to the skin, lips or epidermal derivatives, the first composition comprising

a) at least one liquid fatty phase,

b) a dispersion of at least one polymer particle dispersed in the at least one liquid fatty phase, and

c) at least one compound chosen from esters of aliphatic and aromatic polycarboxylic acids and aliphatic and aromatic alcohols comprising from 1 to 10 carbon atoms, the at least one compound being present in an amount sufficient to plasticize the at least one polymer particle,

and the second composition comprising a physiologically acceptable medium.

In one embodiment, the inventor conceived of the manufacture of makeup compositions comprising at least one polymer particle dispersed and surface-stabilized by at least one stabilizer in a liquid fatty phase, such as those described in EP 1 249 223 (L'Oréal).

In one embodiment, the inventor has found that selecting a particular plasticizer with selected solubility parameters provides compositions, comprising at least one polymer particle dispersed in a liquid fatty phase, whose staying power and/or transfer resistance and/or ease of application are enhanced. Examples of plasticizers include those referred to in FR-A-2 782 917.

For example, the product disclosed herein may in some embodiments provide access to continuous, non-tacky deposits which exhibit good coverage, do not migrate and do not dry out the skin or lips to which they are applied, either during application or over time. The product may in some embodiment also provide good application properties: the deposit dries rapidly and the colour is deposited correctly in a single pass. In a further embodiment, the product allows a natural makeup result to be obtained, with well-defined contours, which leaves a sensation of bare lips, in contrast to the prior art compositions, during the deposition of which the consumer is aware of her lips retracting, of unattractive emphasizing of the lip microrelief, and of a sensation of drying.

In some embodiments, the compositions described here may provide the qualities of spreading and of adhesion to the skin, lips, eyelashes or mucosae, and also a pleasant and creamy feel.

In another embodiment, properties of staying power, transfer resistance and migration resistance may be provided which are suitable for producing makeup products for the face, for example, the lips, such as lipstick and lipgloss, or the eyes, such as mascara, eyeliner or eyeshadow, or the skin, such as foundation.

In some embodiments, the invention applies not only to lip makeup products but also to products for caring for and/or treating the lips, such as lip balm, and the skin including the scalp, such as facial sun protection and daycare creams, to skin makeup products, both for the face and for the human body, such as foundations, for example, those cast in the form of sticks or dishes, concealer products and temporary tattooing products, to body hygiene products such as deodorants, such as stick deodorants, and to eye makeup products, such as eyeliners, for example, in pencil form, and mascaras, such as in cake form.

Definitions

A liquid fatty phase as used herein is any non-aqueous medium which is liquid at ambient temperature (25° C.) and atmospheric pressure (760 mm Hg) and is composed of at least one fatty substance which is liquid at ambient temperature, also called oil, with the exception of an ester formed from acid and polyol, and gelling agents and stabilizers for pigments optionally present in the composition, provided that they are liquid at ambient temperature and atmospheric pressure. This fatty phase may comprise a volatile liquid fatty phase and/or a non-volatile fatty phase.

A non-volatile fatty phase is any medium apt to remain on the skin or lips without evaporating. A non-volatile fatty phase has, for instance, a non-zero vapour pressure at ambient temperature and atmospheric pressure, such as less than 0.02 mm Hg and, for example, less than 10⁻³ mm Hg.

A volatile fatty phase is any non-aqueous medium apt to evaporate from the skin or lips at ambient temperature and atmospheric pressure. This volatile phase may comprise, for example, oils having a vapour pressure at ambient temperature (25° C.) and atmospheric pressure (760 mm Hg) ranging from 10⁻³ to 300 mm Hg (0.13 Pa to 40,000 Pa), such as ranging from 0.02 to 300 mm Hg (2.66 Pa to 40,000 Pa).

The term “goniochromatic” means able to produce different colors depending on the light incidence and viewing angle.

The term “hydrocarbon oil” refers to oils containing primarily carbon atoms and hydrogen atoms, for example, alkyl or alkenyl chains, such as alkanes or alkenes, and also oils having an alkyl or alkenyl chain comprising at least one group chosen from ethers, esters and carboxylic acids.

The term “substantially free” means not more than trace amounts or impurities.

The term “substituted” means further comprising at least one substituent chosen from an oxygen, nitrogen and halogen atom and a hydroxyl, ether, oxyalkylene, polyoxyalkylene, carboxyl, amine and amide group.

The term “saccharide” refers to a monosaccharide or a polysaccharide.

“Polar groups” are well known to the person skilled in the art; they may be, for example, ionic or non-ionic polar groups chosen from —COOH; —OH; ethylene oxide; propylene oxide; —PO₄; —NHR; and —NR₁R₂, wherein R₁ and R₂ may form a ring and may be chosen from linear and branched C₁ to C₂₀ alkyl and alkoxy radicals.

A gelling agent is a compound which increases the viscosity of the medium into which it is incorporated or which stiffens the medium. The gelling agents of the present disclosure do not include waxes.

A wax as used herein is a lipophilic fatty compound which is solid at ambient temperature (25° C.) and exhibits a reversible solid/liquid state change, the compound having a melting temperature of greater than 40° C. and possibly up to 200° C. and having an anisotropic crystalline organization in the solid state. The size of the crystals is such that they diffract and/or scatter light, thereby endowing the composition with a turbid, opaque appearance. By bringing the wax to its melting temperature it can be made miscible with the oils and made to form a microscopically homogeneous mixture; however, by bringing the temperature of the mixture back to ambient temperature, the wax is recrystallized in the oils of the mixture. In one embodiment, it is this recrystallization in the mixture which is responsible for the decrease in the gloss of the mixture.

The “at least one compound being present in an amount sufficient to plasticize the at least one polymer particle” is an organic compound which is present in an amount sufficient to lower the glass transition temperature of the at least one polymer particle disclosed herein by at least 2° C., such as at least 3° C. or at least 4° C., for example, by at least 5° C.

The glass transition temperature of the at least one polymer particle disclosed herein is measured using a differential scanning calorimeter, for example, the calorimeter sold under the name MDSC 2920 by the company TA Inst., by the technique referred to as differential scanning calorimetry (DSC), in which, for example, a temperature rise of 2° C. per minute is employed.

In order to evaluate the reduction in glass transition temperature caused by the compound the starting point is to measure the glass transition temperature of the pure polymer particle by the above method and then to prepare a mixture of the at least one polymer particle and the at least one compound and to carry out another measurement by the same method under the same conditions.

The reduction in glass transition temperature is a function of the amount of plasticizing compound in the composition. This quantity must be sufficient to bring about a reduction, an effective decrease in the glass transition temperature of the at least one polymer particle. This reduction or decrease is at least 2° C., for example, at least 4° C., such as at least 5° C.

### SUMMARY OF THE INVENTION

The disclosure first provides a cosmetic composition intended for application to keratin materials, comprising

a) at least one liquid fatty phase,

b) a dispersion of at least one polymer particle dispersed in the at least one liquid fatty phase, and

c) at least one compound having a solubility parameter, δₕ, ranging from 5.5 to 11 (J/cm³)^{1/2} and chosen from esters of aliphatic and aromatic polycarboxylic acids and aliphatic and aromatic alcohols comprising from 1 to 10 carbon atoms, the at least one compound being present in an amount sufficient to plasticize the at least one polymer particle.

Another non-limiting embodiment of the cosmetic composition disclosed herein intended for application to keratin materials, comprises a) at least one liquid fatty phase, b) particles of at least one polymer, dispersed in the liquid fatty phase, and c) at least one compound chosen from esters of aliphatic and aromatic polycarboxylic acids and aliphatic and aromatic alcohols comprising from 1 to 10 carbon atoms, the compound being present in an amount sufficient to plasticize the at least one polymer particle.

The disclosure further provides for a method of making a composition intended for application to the skin, the lips and the epidermal derivatives, the composition being cosmetic or hygienic, or for preparing a dermatological or pharmaceutical composition for application to the skin, lips and epidermal derivatives, comprising adding in the composition particles of at least one polymer dispersed in a liquid fatty phase and at least one compound chosen from esters of aliphatic and aromatic polycarboxylic acids and aliphatic and aromatic alcohols comprising from 1 to 10 carbon atoms, the compound being present in an amount sufficient to plasticize the at least one polymer particle, in order to limit the transfer of the composition and/or to increase its staying power over time and/or to enhance its deposition.

The disclosure additionally provides a cosmetic method of caring for or making up keratin materials, such as the lips, epidermal derivatives or skin, which consists in applying to the lips, epidermal derivatives or skin the cosmetic composition disclosed herein.

The disclosure further provides a method of limiting the transfer of a lip or skin care or makeup composition and/or increasing its staying power over time and/or facilitating its application, the composition comprising at least one liquid fatty phase and at least one polymer particle dispersed in the fatty phase, the method comprising introducing into the fatty phase at least one compound chosen from esters of polycarboxylic aliphatic and aromatic acids and aliphatic and aromatic alcohols comprising from 1 to 10 carbon atoms, the compound being in an amount sufficient to plasticize the at least one polymer particle.

The disclosure additionally provides a method of producing a cosmetic composition, which comprises introducing into a physiologically acceptable liquid medium a) a dispersion of polymer particles in a liquid fatty phase and b) at least one compound chosen from esters of aliphatic and aromatic polycarboxylic acids and aliphatic and aromatic alcohols comprising from 1 to 10 carbon atoms, the compound being in an amount sufficient to plasticize the polymer.

Polymer Particles in dispersion

The at least one polymer particle disclosed herein may be a solid which is insoluble in the liquid organic phase of the first composition even at its softening temperature, in contradistinction to a wax, even a wax of polymeric origin, which is soluble in the liquid organic phase (or fatty phase) at its melting temperature. In addition, it may allow the formation of a deposit, such as a film-forming deposit, which may be continuous and homogeneous and/or is characterized by the interpenetration of the polymeric chains. With a wax, even one obtained by polymerization, melting in the liquid organic phase may be followed by recrystallization. It is this recrystallization which may be responsible, for the loss of gloss of the composition.

In one embodiment, to optimize the non-transfer properties the amount of polymer particles is selected as a function of the amount of colorants and/or actives and/or oils present in the first composition. As disclosed herein the amount of polymer particles may be greater than 5% by weight (in terms of active substance) relative to the total weight of the composition.

In one embodiment, a dispersion of polymer particles is used in the composition disclosed hereinbecausethese particles remain in the state of elementary particles, without forming agglomerates, in the fatty phase. The polymer particle dispersion imay also provide the possibility of obtaining highly fluid compositions (of the order of 130 centipoises), even in the presence of a high proportion of polymer.

When using a polymer dispersion of this kind, the polymer particle size may be calibrated at will and their size “polydispersity” modified during the synthesis. It may be possible to obtain very small-sized particles which are invisible to the naked eye when in the composition and when applied to the skin, lips or epidermal derivatives.

One composition of the product of the disclosure may comprises at least one stable dispersion of polymer particles, generally spherical particles, of at least one polymer particle in a physiologically acceptable liquid organic phase. These dispersions may, for example, be in the form of polymer nanoparticles in stable dispersion in the liquid organic phase. The nanoparticles may be of an average size, for example, ranging from 5 to 800 nm, such as from 50 to 500 nm. It is possible, however, to obtain polymer particle sizes ranging up to 1 μm.

The at least one polymer particle in dispersion may be, for example, insoluble in water-soluble alcohols such as ethanol, for example.

The at least one polymer particle in dispersion that may be used in the first composition of the invention may have, for example, a molecular weight ranging from 2,000 to 10,000,000 g/mol.

It is possible to use filmable polymer particles, such as those having a low glass transition temperature, T_{g}, for example, less than or equal to the temperature of the skin, such as less than or equal to 40° C.

The at least one polymer particle used may be filmable, that is, it is able, alone or in combination with a plasticizer, to form an isolatable film. It is also possible to use a non-filmable polymer particle.

A non-filmable polymer particle is a polymer particle which is not capable on its own of forming an isolatable film. In combination with a non-volatile compound of the oil type, this polymer particle allows a continuous and homogeneous deposit to be formed on the skin and/or lips.

Among filmable polymer particles mention may be made of radical, acrylic or vinyl homopolymers or copolymers, such as those having a T_{g} of less than or equal to 40° C., for example, ranging from −10° C. to 30° C., which are used alone or in a mixture.

Among non-filmable polymer particles mention may be made of radical, vinyl or acrylic homopolymers or copolymers, optionally crosslinked, such as those having a T_{g} of greater than 40° C., for example, ranging from 45° C. to 150° C., which are used alone or in a mixture.

A radical polymer is a polymer obtained by polymerizing monomers comprising unsaturation, for example, ethylenic unsaturation, each monomer being capable of undergoing homopolymerization (in contrast to polycondensates). The radical polymers may be, for example, vinyl polymers or copolymers, such as acrylic polymers.

The acrylic polymers may result from the polymerization of ethylenically unsaturated monomers having at least one acid group and/or esters of these acid monomers and/or amides of these acids.

As a monomer carrying an acid group it is possible to use α,β-ethylenic unsaturated carboxylic acids such as acrylic acid, methacrylic acid, crotonic acid, maleic acid and itaconic acid. For example, (Meth)acrylic acid and crotonic acid may be used, and, for further example, (meth)acrylic acid.

The esters of acid monomers may be chosen from esters of (meth)acrylic acid (also called (meth)acrylates), such as alkyl (meth)acrylates, for example, C₁-C₂₀, such as C₁-C₈, alkyl (meth)acrylates, aryl (meth)acrylates, for example, C₆-C₁₀ aryl (meth)acrylates, and hydroxyalkyl (meth)acrylates, for example, C₂-C₆ hydroxyalkyl (meth)acrylates. Alkyl (meth)acrylates include methyl, ethyl, butyl, isobutyl, 2-ethylhexyl and lauryl (meth)acrylate. Hydroxyalkyl (meth)acrylates include hydroxyethyl (meth)acrylate and 2-hydroxypropyl (meth)acrylate. Aryl (meth)acrylates include benzyl acrylate and phenyl acrylate.

For example, the esters of (meth)acrylic acid such as the alkyl (meth)acrylates may be used.

As the radical polymer copolymers of (meth)acrylic acid and alkyl (meth)acrylate, such as C₁-C₄ alkyl (meth)acrylate, may be used. For example, It is possible to use methyl acrylates optionally copolymerized with acrylic acid.

Amides of the acid monomers include (meth)acrylamides, for example, N-alkyl (meth)acrylamides, for further example, N-C₂-C₁₂-alkyl(meth)acrylamides, such as N-ethylacrylamide, N-t-butylacrylamide and N-octylacrylamide, and N-di(C₁-C₄)alkyl(meth)acrylamides.

The acrylic polymers may result from the polymerization of ethylenically unsaturated monomers having at least one amine group, in free form or else in partly or totally neutralized form, or else in partly or totally quaternized form. Such monomers may be, for example, dimethylaminoethyl (meth)acrylate, dimethylaminoethylmethacrylamide, vinylamine, vinylpyridine or diallyldimethylammonium chloride.

The vinyl polymers may result from the homopolymerization or copolymerization of at least one monomer chosen from vinyl esters and styrenic monomers. For example, these monomers may be polymerized with acid monomers and/or their esters and/or their amides, such as those mentioned above. Non-limiting examples of vinyl esters include vinyl acetate, vinyl propionate, vinyl neodecanoate, vinyl pivalate, vinyl benzoate and vinyl t-butylbenzoate. Styrenic monomers include, for example, styrene and alpha-methylstyrene.

The list of monomers given is not limiting and it is possible to use any monomer known to the person skilled in the art which falls within the categories of acrylic monomers and vinyl monomers (including monomers modified with a silicone chain).

As other vinyl monomers which may be used non-limiting mention may also be made of the following:

N-vinylpyrrolidone, vinylcaprolactam, vinyl-N-(C₁-C₆)alkylpyrroles, vinyloxazoles, vinylthiazoles, vinylpyrimidines and vinylimidazoles; and

olefins such as ethylene, propylene, butylene, isoprene and butadiene.

The vinyl polymer may be crosslinked using at least one difunctional monomer, comprising, for example, at least two ethylenic unsaturations, such as ethylene glycol dimethacrylate or diallyl phthalate.

Without limitation, the polymer particles in dispersion disclosed herein may be chosen from the following polymers and copolymers: polyurethanes, acrylic polyurethanes, polyureas, polyu rea-polyurethanes, polyester-polyu rethanes, polyether-polyurethanes, polyesters, polyester amides, fatty-chain polyesters, alkyds; acrylic and/or vinyl polymers or copolymers; acrylic-silicone copolymers; polyacrylamides; silicone polymers such as silicone polyurethanes or silicone acrylics, fluorinated polymers, and mixtures thereof.

The at least one polymer particle in dispersion in the organic liquid phase may range from 5 to 40% by weight of the dry-matter content of the composition, for example, from 5 to 35% such as from 8 to 30%.

In one non-limiting embodiment the polymer particles in dispersion are surface-stabilized by at least one stabilizer which is solid at ambient temperature. In that case the amount of the dispersion in terms of dry matter represents the total amount of polymer and stabilizer, subject to the proviso that the amount of dry matter of polymer may not be less than 5%.

One choice is to use a dispersion of filmable polymer particles, the particles being dispersed in a volatile oil.

Plasticizing the polymer

The at least one polymer particle in dispersion which can be used in the first composition of the invention may, for instance, have a glass transition temperature (T_{g}) ranging from −100° C. to 300° C., such as from −50° C. to 100° C., for example, from −10° C. to 50° C.

In the context of the present disclosure a sufficient amount of at least one compound is added to the at least one polymer particle in dispersion so as to lower the T_{g} of the polymer particle film and to increase the adhesion of the polymer particle film to its substrate, such as the keratin materials. The plasticizing compound may lower the glass transition temperature of the at least one polymer particle by at least 2, 3, or 4° C., for example, from 5° C. to 20° C. In one non-limiting embodiment the plasticizer compound lowers the glass transition temperature of the at least one polymer particle by at least 2, 3 or 4° C., for example, from 5° C. to 20° C., when the plasticizing compound represents not more than 10% by weight of the at least one polymer particle.

In one non-limiting embodiment the compound has a solubility parameter, δₕ, ranging from 5.5 to 11, for example, from 5.9 to 11, such as from 7 to 10.5, for further example from 8 to 10, such as from9 to 10 (J/cm³)^{1/2}.

The solubility parameter δₚ of the compound may be, for example, from 1.5 to 4.5, such as from 1.5 to 4, for example, from 1.5 to 3.5, such as from 2 to 3 (J/cm³)^{1/2}.

The compound may be, for example, liquid at ambient temperature and atmospheric pressure. The compound may be, for example, tributoxyethyl phosphate.

The definition of the solubility parameters according to Hansen is well known to the person skilled in the art and is described in particular in the article by C. M. Hansen: “The three dimensional solubility parameters” J. Paint Technol. 39, 105 (1967). These parameters are also described in JP-A-08-109121 from Kao and in the document by D. W. Van Krevelen “Properties of polymers” (1990), p. 190.

According to Hansen:

δₚ characterizes the Debye interaction forces between permanent dipoles; and

δₕ characterizes the specific interaction forces (of hydrogen bond, acid/base, donor/receptor type, etc.).

The parameters δₚ and δₕ are generally expressed in (J/cm³)^{1/2}. They are determined at ambient temperature (25° C.) and may be calculated, for example, according to the method disclosed in the above Kao patent.

In the composition disclosed herein it is possible to use a mixture of compounds satisfying the above conditions. In this case the solubility parameters of the mixture are determined from those of the compounds taken separately, according to the following relationships:

$${δ}_{pmix} = {\sum }_{i} x i {δ}_{p i} and {δ}_{hmix} = {\sum }_{i} x i {δ}_{h i}$$

where xi represents the volume fraction of the compound (i) in the mixture.

It is within the scope of the person skilled in the art to determine the amounts of each compound so as to obtain a mixture of compounds meeting the above conditions.

In this non-limiting embodiment the compound may be selected from esters of at least one carboxylic acid comprising from 1 to 7 carbon atoms and a polyol comprising at least 4 hydroxyl groups.

The polyol disclosed herein may be a monosaccharide —a polyhydroxyaldehyde (aldose) or polyhydroxyketone (ketose)—which may be cyclized or not. The polyol may be, for example, a monosaccharide cyclized in hemiacetal form.

The polyol may also be a polyol derived from a monosaccharide, such as erythritol, xylitol or sorbitol.

Among aldoses, mention may be made of D-ribose, D-xylose, L-arabinose, D-glucose (or alpha-D-glucopyranose when in cyclic hemiacetal form), D-mannose and D-galactose.

Among ketoses, mention may be made of D-xylulose and D-fructose (or beta-D-fructofuranose when in cyclic hemiacetal form).

The polyol may be a monosaccharide or a polysaccharide comprising from 1 to 10 monosaccharide units, for example, from 1 to 4, such as 1 or 2 monosaccharide units. The polyol may be chosen from erythritol, xylitol, sorbitol, glucose and sucrose.

The polyol disclosed herein may be, for example, a disaccharide. Among disaccharides, mention may be made of sucrose (alpha-D-glucopyranosyl-(1-2)-beta-D-fructofuranose), lactose (beta-D-galactopyranosyl-(1-4)-beta-D-glucopyranose) and maltose (alpha-D-glucopyranosyl-(1-4)-beta-D-glucopyranose).

The polyol may be a polysaccharide comprising two or more identical monosaccharide units or at least two different monosaccharide units. The ester disclosed herein may comprise a polyol substituted by at least two different monocarboxylic acids or by at least three different monocarboxylic acids.

The ester may be obtained by copolymerizing two esters disclosed herein, for example, by copolymerizing i) a sucrose substituted by benzoyl groups and ii) a sucrose substituted by acetyl and/or isobutyryl groups.

The acid may be, for instance, a monocarboxylic acid chosen from, for example, acids comprising from 1 to 7 carbon atoms, for further example, 1 to 5 carbon atoms, such as acetic, n-propanoic, isopropanoic, n-butanoic, isobutanoic, tert-butanoic, n-pentanoic and benzoic acids.

The ester may be obtained from at least two different monocarboxylic acids. In one non-limiting embodiment the acid may be chosen froman unsubstituted linear and branched acid.

The acid may be, for example, chosen from acetic acid, isobutyric acid and benzoic acid.

In one non-limiting embodiment the ester is sucrose diacetate hexa(2-methylpropanoate).

In another non-limiting embodiment the compound disclosed herein may be chosen from esters of aliphatic and aromatic polycarboxylic acids and aliphatic and aromatic alcohols comprising from 1 to 10 carbon atoms.

The aliphatic and aromatic alcohols disclosed herein comprise from 1 to 10 carbon atoms, for example, 1 to 8, such as 1 to 6. They may be chosen from alcohols R1OH such that R1 is chosen from methyl, ethyl, propyl, isopropyl, butyl, hexyl, ethylhexyl, decyl, isodecyl, and benzyl substituted by an alkyl comprising from 1 to 4 carbon atoms.

The aliphatic and aromatic polycarboxylic acids may comprise, for example, 3 to 12 carbon atoms, such as 3 to 10 carbon atoms, for example, 3 to 8 carbon atoms, such as 6 or 8 carbon atoms.

The aliphatic and aromatic polycarboxylic acids may be dicarboxylic acids or tricarboxylic acids.

Among dicarboxylic acids mention may be made of those corresponding to the formula HOOC—(CH₂)ₙ—COOH such that n ranges from 1 to 10, such as from 2 to 8, for example, 2, 4, 6 or 8.

For instance, the dicarboxylic acids may be chosen from succinic, adipic and sebacic acids.

The dicarboxylic acids may also be chosen from phthalic acid and its derivatives, for example, butyl benzyl phthalate, dibutyl phthalate, diethylhexyl phthalate, diethyl phthalate and dimethyl phthalate.

Among tricarboxylic acids mention may be made of the triacids corresponding to the formula

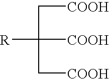

wherein R is a hydrocarbon radical, for example H, —OH or —OCOR′ such that R′ is an alkyl such as an alkyl comprising from 1 to 6 carbon atoms.

The tricarboxylic acids may be chosen, for example, from acetylcitric acid and its derivatives.

Among the citrates mention may be made of tributyl acetylcitrate, triethyl acetylcitrate, triethylhexyl acetylcitrate, trihexyl acetylcitrate, trihexyl butyroylcitrate, isodecyl citrate, isopropyl citrate, tributyl citrate and triethylhexyl citrate. In one non-limiting embodiment the carboxylic acid is not tributyl acetylcitrate.

Among adipates mention may be made of dibutyl adipate and di-2-ethylhexyl adipate. In one non-limiting embodiment the compound disclosed herein is not diisopropyl adipate.

Among sebacates mention may be made of dibutyl sebacate, diethylhexyl sebacate, diethyl sebacate and diisopropyl sebacate.

Among succinates mention may be made of diethylhexyl succinate and diethyl succinate.

The compound may be chosen from compounds having a molecular mass of less than 5,000, for example, less than 2,000, such as less than 1,000, for further example, less than 900 g/mol. The molecular mass of the compound may be greater than 100 g/mol.

The compound may comprise no polar group, for example, no hydroxyl group. The polar groups may be, for example, ionic and non-ionic polar groups chosen from —COOH; —OH; ethylene oxide; propylene oxide; —PO₄; —NHR; —NR₁R₂ where R₁ and R₂, which may identical or different, are chosen from linear and branched C₁ to C₂₀ alkyls and alkoxy radicals and optionally form a ring.

The compound may be present, for example, in an amount ranging from 0.1 to 25% by weight, for further example,from 0.5 to 15% by weight, such as 3 to 15% by weight, relative to the total weight of the composition.

The mass ratio between the at least one polymerparticle and the compound may be, for example, from 0.5 to 100, for further example, from 1 to 50, suchas from 1 to 10, such as from 1 to 5.

Polymer particle stabilization

The at least one polymer particle may be, for example, surface-stabilized in the at least one liquid fatty phase by a stabilizer which may be a polymer chosen from a block polymers, graft polymers and random polymers. Stabilization can be effected by any known means, such as by direct addition of the block polymer, graft polymer and/or random polymer during the polymerization.

The stabilizer may be present, for example, in the mixture before polymerization of the polymer. However, it is also possible to add it continuously, such as when the monomers are also added continuously.

The stabilizer may be used in an amount ranging from 2 to 30% by weight, relative to the initial mixture of monomers, such as from 5 to 20% by weight.

In one embodiment, when a graft polymer and/or block polymer is used as the stabilizer, the synthesis solvent is chosen such that at least some of the grafts or blocks in the stabilizing polymer are soluble in the solvent, and the remainder of the grafts or blocks are not soluble therein. The stabilizing polymer used during the polymerization should be soluble or dispersible in the synthesis solvent. Furthermore, the stabilizer may bechosen, for example, whose insoluble blocks or grafts have a certain affinity for the polymer formed during the polymerization.

Among graft polymers mention may be made of silicone polymers grafted with a hydrocarbon chain and hydrocarbon polymers grafted with a silicone chain.

Thus, block or graft block copolymers comprising at least one block of polyorganosiloxane type and at least one block of a radical polymer can be used, such as graft copolymers of acrylic/silicone type, which can be used, for example, when the non-aqueous medium is silicone-based.

It is also possible to use block or graft block copolymers comprising at least one block of polyorganosiloxane type and at least one polyether. The polyorganopolysiloxane block can be, for example, a polydimethylsiloxane or alternatively a poly(C₂-C₁₈)-alkylmethylsiloxane; the polyether block can be a poly(C₂-C₁₈)alkylene, such as those chosen from polyoxyethylene and polyoxypropylene. For example, it is possible to use dimethicone copolyols or (C₂-C₁₈)alkyldimethicone copolyols such as those sold under the name Dow Corning 3225C by the company Dow Corning, and lauryl methicones such as those sold under the name Dow Corning Q2-5200 by the company Dow Corning.

Block or graft block copolymers which may also be mentioned are those comprising at least one block resulting from the polymerization of at least one ethylenic monomer comprising at least one optionally conjugated ethylenic bond, such as ethylenes and dienes such as butadiene and isoprene, and at least one block of a vinyl polymer such as a styrenic polymer. When the ethylenic monomer comprises two or more optionally conjugated ethylenic bonds, the residual ethylenic unsaturations after the polymerization are generally hydrogenated. Thus, in a known manner, the polymerization of isoprene leads, after hydrogenation, to the formation of ethylene-propylene block, and the polymerization of butadiene leads, after hydrogenation, to the formation of ethylene-butylene block. Among these polymers mention may be made of block copolymers, for example, diblock and triblock type copolymers such as polystyrene/polyisoprene (SI), polystyrene/polybutadiene (SB), such as those sold under the name Luvitol HSB by BASF, polystyrene/copoly(ethylene-propylene) (SEP), such as those sold under the name Kraton by Shell Chemical Co., and polystyrene/copoly(ethylene-butylene) (SEB). For example, Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS) and Kraton D-1107 (SIS) may be used. The polymers are generally referred to as copolymers of hydrogenated or non-hydrogenated dienes.

Gelled Permethyl 99A-750, 99A-753-59 and 99A-753-58 (mixture of triblock polymer and star polymer), Versagel 5960 from Penreco (triblock polymer+star polymer), and OS129880, OS129881 and OS84383 from Lubrizol (styrene/methacrylate copolymer) can also be used.

As block or graft block copolymers comprising at least one block resulting from the polymerization of at least one ethylenic monomer comprising at least one ethylenic bond and at least one block of an acrylic polymer, mention may be made of poly(methyl methacrylate)/polyisobutylene diblock and triblock copolymers and graft copolymers comprising a poly(methyl methacrylate) backbone and polyisobutylene grafts.

As block or graft block copolymers comprising at least one block resulting from the polymerization of at least one ethylenic monomer comprising at least one ethylenic bond and at least one block of a polyether such as a C₂-C₁₈ polyalkylene (in particular polyethylenated and/or polyoxypropylenated), mention may be made of polyoxyethylene/polybutadiene and polyoxyethylene/polyisobutylene diblock and triblock copolymers.

In another embodiment, when a random polymer is used as a stabilizer, it is selected such that it has a sufficient amount of groups making it soluble in the intended synthesis solvent.

It is thus possible to use copolymers based on alkyl acrylates or methacrylates derived from C₁-C₄ alcohols and alkyl acrylates and methacrylates derived from C₈-C₃₀ alcohols. Mention may be made, for example, of stearyl methacrylate/methyl methacrylate copolymer.

In one embodiment, when the polymer synthesis solvent is apolar, a polymer may be chosen, for example, which gives the most complete coverage possible of the particles, a plurality of stabilizing polymer chains then being adsorbed onto a polymer particle obtained by polymerization.

In this case, either a graft polymer or a block polymer may be used, for example, as a stabilizer, so as to have better interfacial activity. The reason for this is that blocks or grafts which are insoluble in the synthesis solvent give a more voluminous coverage at the surface of the particles.

When the liquid synthesis solvent comprises at least one silicone oil, the stabilizer may be chosen from, for example, block and graft block copolymers comprising at least one block of polyorganosiloxane type and at least one block chosen from a radical polymer,a polyether, and a polyester such as polyoxypropylenated and/or oxyethylenated blocks.

When the liquid fatty phase comprises no silicone oil, the stabilizer may be, for example, selected from the group consisting of:

(a) block and graft block copolymers comprising at least one block of polyorganosiloxane type and at least one block chosen from radical polymers, polyethers, and polyesters,

(b) copolymers of alkyl acrylates and methacrylates derived from C₁-C₄ alcohols, and of alkyl acrylates and methacrylates derived from C₈-C₃₀ alcohols,

(c) block and graft block copolymers comprising at least one block resulting from the polymerization of at least one ethylenic monomer comprising conjugated ethylenic bonds, and at least one block chosen from vinyl polymers, acrylic polymers, polyethers, polyesters, and mixtures thereof.

Diblock polymers may be used, for example, as a stabilizer.

Fatty phase

The liquid fatty phase of the composition may, for example, be comprised of any cosmetically or dermatologically acceptable, and more generally physiologically accpetable, oil, selected, for example, from oils of mineral, vegetable or synthetic origin, including carbon-based oils, hydrocarbon oils, fluoro oils and silicone oils, alone or in a mixture, insofar as they form a homogeneous and macroscopically stable mixture and as they are compatible with the intended use.

The total liquid fatty phase of the composition may range from 5 to 90% of the total weight of the composition, for example, from 20 to 85%. It represents at least 30% of the total weight of the composition. This fatty phase may comprise, for example, at least one volatile oil.

Volatile oils of the fatty phase

In one embodiment, the compositions comprise at least one volatile oil.

These oils, for example, may be hydrocarbon oils and silicone oils optionally comprising groups chosen from alkyl groups and alkoxy groups, pendantly or at the end of the silicone chain.

As volatile silicone oils which can be used in the disclosure mention may be made of linear and cyclic silicones having a viscosity at ambient temperature of less than 8 cSt and having, for example, 2 to 7 silicon atoms, these silicones optionally comprising groups chosen from alkyl groups and alkoxy groups having 1 to 10 carbon atoms. As volatile silicone oils which can be used in the disclosure mention may be made, for example,of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane and mixtures thereof.

Other volatile oils which can be used in the disclosure include, for example, C₈-C₁₆ isoalkane oils (also called isoparaffins) such as isododecane, isodecane, isohexadecane and, for example, the oils sold under the trade names Isopar and Permethyl, such as isododecane (Permethyl 99A).

The volatile oils range from 5 to 85% of the total weight of the composition, for example, from 20 to 75%.

Synthesis solvent of the polymer particles

The polymer dispersion may be prepared as described in EP-A-749 747.

In one embodiment, a mixture is prepared comprising the initial monomers and a free-radical initiator. This mixture is dissolved in a solvent which, for the remainder of the present disclosure, is referred to as the “synthesis solvent”. When the fatty phase is a non-volatile oil, the polymerization may be carried out in an apolar organic solvent (synthesis solvent) and then the non-volatile oil (which must be miscible with the synthesis solvent) is added and the synthesis solvent is distilled off selectively.

A synthesis solvent may be selected such that the initial monomers and the free-radical initiator are soluble therein and the polymer particles obtained are insoluble therein, so that they precipitate from the solvent as they are formed. The synthesis solvent may, for example, be chosen from alkanes such as heptane, isododecane and cyclohexane.

When the fatty phase selected is a volatile oil the polymerization may be carried out directly in the oil, which hence also acts as the synthesis solvent. The monomers must also be soluble therein, and the free-radical initiator too, and the polymer obtained must be insoluble therein.

The monomers are, for example, present in the synthesis solvent, prior to polymerization, in an amount ranging from 5 to 20% by weight of the reaction mixture. The total amount of the monomers may be present in the solvent before the beginning of the reaction, or a fraction of the monomers may be added at the rate at which the polymerization reaction proceeds.

The free-radical initiator may be, for example, azobisisobutyronitrile or tert-butyl peroxy-2-ethylhexanoate.

The volatile phase of the composition may comprise the synthesis solvent of the dispersed polymer particles.

Non-volatile oil of the fatty phase

The fatty phase may comprises at least one oil chosen from apolar and relatively non-polar non-volatile oil.

As relatively non-polar non-volatile oils which can be used in the disclosure mention may be made of apolar oils and oils containing an alkyl chain such as a C₃-C₄₀ alkyl chain. Examples of apolar and relatively non-polar oils include the following:

linear and branched hydrocarbons such as liquid paraffin, vaseline oil and light naphthalene oil and hydrogenated polyisobutene;

hydrocarbon oils of animal origin such as squalene;

vegetable hydrocarbon oils such as the liquid triglycerides of fatty acids of at least 10 carbon atoms;

synthetic esters and ethers, for example, those of fatty acids, such as the oils of formula R₁(CO)ₓOR₂ wherein R₁ is the residue of an acid comprising 2 to 29 carbon atoms; x is 0 or 1; and R₂ is a hydrocarbon chain comprising 3 to 30 carbon atoms, such as, for example, tributyl acetylcitrate, oleyl erucate, 2-octyldodecyl behenate, triisoarachidyl citrate, isocetyl stearoylstearate or octyldodecanyl stearoylstearate, n-propyl acetate, tridecyl trimellitate, dodecane dioleate or diisocetyl stearate, arachidyl propionate, dibutyl phthalate, propylene carbonate and octyldodecyl pentanoate; polyol esters such as vitamin F, sorbitan isostearate, glyceryl triisostearate and diglycerol;

silicone oils such as polydimethylsiloxanes (PDMS), optionally comprising a chain chosen from C₃-C₄₀ alkyl, C₃-C₄₀ alkoxy, and a phenyl chain, such as phenyltrimethicones, polyalkylmethylsiloxanes, optionally fluorinated such as the polymethyltrifluoropropyldimethylsiloxanes, or optionally with functional groups such as hydroxyl, thiol and amine groups; and polysiloxanes modified with at least one of fatty acids, fatty alcohols and polyoxyalkylenes;

fluoro oils; and

mixtures thereof.

These non-volatile apolar or relatively non-polar oils may range from 0.1 to 20% of the total weight of the composition, for example, from 0.5 to 10%, such as from 1 to 5% of the total weight of the composition.

The non-volatile oil may be, for example, apolar. It may be chosen from hydrocarbons, for example, alkanes, such as hydrogenated polyisobutene.

The fatty phase may further comprise a polar oil chosen from fatty acid esters of 7 to 29 carbon atoms such as diisostearyl malate, isopropyl palmitate, diisopropyl adipate, the triglycerides of caprylic/capric acids, such as those sold by Stearineries Dubois or those sold under the names Miglyol 810, 812 and 818 by Dynamit Nobel, the oil of shea butter, isopropyl myristate, butyl stearate, hexyl laurate, diisopropyl adipate, isononyl isononanoate, 2-hexyldecyl laurate, 2-octyidecyl palmitate, 2-octyidodecyl myristate or lactate, di-2-ethylhexyl succinate, 2-ethylhexyl palmitate, 2-octyldodecyl stearate, castor oil; esters of lanolic acid, lauric acid and stearic acid; higher fatty alcohols (of 7 to 29 carbon atoms) such as stearyl alcohol, linoleyl alcohol or linolenyl alcohol, isostearyl alcohol, 2-octyldodecanol, decanol, dodecanol, octadecanol or oleyl alcohol; higher fatty acids (of 7 to 29 carbon atoms) such as myristic acid, palmitic acid, stearic acid, behenic acid, oleic acid, linoleic acid, linolenic acid or isostearic acid; and mixtures thereof.

These non-volatile polar oils may range from 0.1 to 10% of the total weight of the composition such as from 1 to 5%.

In one non-limiting embodiment of the disclosure the liquid fatty phase comprises at least one apolar volatile oil and at least one apolar non-volatile oil.

Gelling agent

In one embodiment of the disclosure the first composition may comprise a gelling agent for the liquid fatty phase, the agent being chosen from a polymeric and mineral gelling agent.

The gelling agent increases the viscosity of the liquid fatty phase or stiffens it. The gelling agent according to the present disclosure does not embrace waxes.

The gelling agent may also be chosen from ethylene homopolymers and copolymers whose weight-average molecular mass ranges from 300 to 500,000 such as from 500 to 100,000.

The gelling agent may be chosen from olefin copolymers of controlled crystallization, as described in application EP-A-1 034 776 from the inventor, such as, for example, the ethylene/octene copolymer sold under the reference Engage 8400 by Dupont de Nemours.

This olefin copolymer or copolymers is/are used for example at concentrations ranging from 0.5 to 20%, for example, from 1 to 10% of the total weight of the first composition.

In another non-limiting embodiment a polycaprolactone is used as a gelling agent.

The polycaprolactone may be chosen from, for example, ε-caprolactone homopolymers. Homopolymerization may be initiated with a diol, for example, a diol comprising 2 to 10 carbon atoms, such as diethylene glycol, 1,4-butanediol and neopentyl glycol.

It is possible, for example, to use polycaprolactones with a molecular weight ranging from 300 to 2,000, such as those sold under the name Capa® 2125 by Solvay.

The polycaprolactone may be present in the composition in an amount ranging from 0.1 to 30% by weight, relative to the total weight of the composition, for example, from 0.5 to 25% by weight, for further example from 1 to 20% by weight, such as from 3 to 15% by weight.

In another non-limiting embodiment the gelling agent present in the composition according to the disclosure may be an amorphous polymer formed by polymerizing an olefin as described in application EP 1 002 528, the content of which is incorporated into the present application by reference. The olefin may be, for example, an elastomeric ethylenically unsaturated monomer.

As examples of olefins mention may be made of ethylenic hydrocarbon monomers, having, for example, one or two ethylenic unsaturations and comprising 2 to 5 carbon atoms, such as ethylene, propylene, butadiene and isoprene.

An amorphous polymerisapolymerwhich does not have a crystalline form.

The amorphous polymeric gelling agent may be chosen from, for example, a diblock, triblock, multiblock, radial and star copolymer and mixtures thereof, for example, a triblock, multiblock, radial and star copolymer, and mixtures thereof.

Polymeric gelling agents of this kind are described in application US-A-2002/005562 and in U.S. Pat. No. 5,221,534.

The polymeric gelling agent is advantageously an amorphous block copolymer of styrene and an olefin.

The polymeric gelling agent may be, for example, hydrogenated in order to reduce the ethylenic unsaturations which remain after the polymerization of the monomers.

For example, the polymeric gelling agent is an optionally hydrogenated copolymer comprising styrene blocks and ethylene/C₃-C₄ alkylene blocks.

The polymeric gelling agent is advantageously a triblock copolymer, for example, hydrogenated, chosen from styrene-ethylene/propylene-styrene copolymers, styrene-ethylene/butadiene-styrene copolymers, styrene-isoprene-styrene copolymers and styrene-butadiene-styrene copolymers. Triblock polymers are sold, for example, under the names Kraton® G1650E, Kraton® G1652, Kraton® D1101, Kraton® D1102 and Kraton® D1160 by Kraton.

The amorphous polymeric gelling agent may be present in an amount ranging from 0.05% to 5% by weight, relative to the total weight of the composition, for example, ranging from 0.1 % to 3% by weight, such as ranging from 0.2% to 2% by weight.

The polymer particles dispersed in the organic phase and the polymeric gelling agent are advantageously present in the composition in an amount such that the weight ratio of dispersed and surface-stabilized polymer particles to polymeric gelling agent ranges from 10 to 30, for example, from 15 to 25, such as from 18 to 22.

In another non-limiting embodiment the gelling agent may, for example, be chosen from:

optionally modified clays, such as hectorites modified with a C₁₀ to C₂₂ fatty acid ammonium chloride, such as hectorite modified with distearyldimethylammonium chloride;

pyrogenic silica, optionally hydrophobically treated on its surface, with a particle size of less than 10 microns; and mixtures thereof.

As organic-phase gelling agents mention may also be made of the following:

partly or totally crosslinked elastomeric organopolysiloxanes of three-dimensional structure, such as those sold under the name KSG6, KSG16 and KSG18 by Shin-Etsu, Trefil E-505C and Trefil E-506C by Dow Corning, Gransil SR-CYC, SR DMF10, SR-DC556, SR 5CYC gel, SR DMF 10 gel and SR DC 556 gel by Grant Industries, and SF 1204 and JK 113 by General Electric;

galactomannans comprising from one to six, for example, from two to four hydroxyl groups per monosaccharide unit, which are substituted by a saturated or unsaturated alkyl chain, such as guar gum alkylated with C₁ to C₆, for example C₁ to C₃ alkyl chains, such as the ethylated guar having a degree of substitution of from 2 to 3, such as that sold by Aqualon under the name N-Hance-AG;

gums, for example, silicone gums, such as PDMS gums, having a viscosity of more than 100,000 cSt, and mixtures thereof.

Colloidal dispersion

The composition according to the invention comprises, for example, particles which are solid at ambient temperature and are dispersed in the physiologically acceptable medium, and which are introduced into the composition in the form of a colloidal dispersion, also referred to as a “particle paste”, as described in application WO 02/39961, the content of which is incorporated by reference into the present application.

A colloidal dispersion or “particle paste” for the purposes of the invention is a concentrated colloidal dispersion of coated or uncoated particles in a continuous medium, this dispersion being stabilized using a dispersant or, where appropriate, without a dispersant. These particles may be chosen from pigments, nacres, solid fillers and mixtures thereof. These particles may be of any form, such as spherical or elongated such as fibres. They are insoluble in the medium.

In one embodiment, the purpose of the dispersant is to protect the dispersed particles against agglomeration or flocculation. The concentration of dispersant generally used to stabilize a colloidal dispersion ranges from 0.3 to 5 mg/m², for example, from 0.5 to 4 mg/m² of particle surface area. This dispersant may be chosen from a surfactant, an oligomer, a polymer or a mixture of two or more of these, and carries one or more functionalities which have a high affinity for the surface of the particles to be dispersed. For example, they are able to attach physically or chemically to the surface of the pigments. These dispersants additionally exhibit at least one functional group which is compatible with or soluble in the continuous medium. Use is made, for example, of esters of 12-hydroxystearic acid, such as of a C₈ to C₂₀ fatty acid and a polyol, such as glycerol or diglycerol, examples being poly(12-hydroxystearic) stearate with a molecular weight of approximately 750 g/mol, such as that sold under the name Solsperse 21 000 by Avecia, polyglyceryl-2 dipolyhydroxystearate (CTFA name), sold under the reference Dehymuls PGPH by Henkel, and also polyhydroxystearic acid, such as that sold under the reference Arlacel P100 by Uniqema, and mixtures thereof.

As other dispersants which can be used in the composition of the disclosure mention may be made of quaternary ammonium derivatives of polycondensed fatty acids, such as Solsperse 17 000 sold by Avecia and the polydimethylsiloxane/oxypropylene mixtures such as those sold by Dow Corning under the references DC2-5185 and DC2-5225 C.

Polydihydroxystearic acid and the esters of 12-hydroxystearic acid are, for example, intended for a hydrocarbon-based or fluorine-based medium, while the dimethylsiloxane oxyethylene/oxypropylene mixtures are intended, for example, for a silicone-based medium.

The colloidal dispersion is a suspension of particles generally of micron size (<10 μm) in a continuous medium. The volume fraction of particles in a concentrated dispersion ranges from 20% to 40%, for example, from 30% to 40%, corresponding to a weight fraction which can range up to 70% depending on the density of the particles.

The particles dispersed in the medium may comprise particles chosen from mineral particles and organic particles and mixtures thereof as described below.

The continuous medium of the paste may be any medium and may comprise any solvent or liquid fatty substance and mixtures thereof. The liquid medium of the particle paste may be one of the liquid fatty substances or oils which it is desired to use in the composition, hence forming part of the liquid fatty phase.

The “particle paste” or colloidal dispersion may be a “pigment paste” comprising a colloidal dispersion of coloured, coated or uncoated, particles. These coloured particles are pigments, nacres or a mixture of pigments and/or nacres.

The colloidal dispersion may range in amount from 0.5 to 60% by weight of the composition, for example, from 2 to 40%, such as from 2 to 30%.

The pigments may be white or coloured pigments, mineral and/or organic pigments, interference pigments or non-interference pigments. Among mineral pigments mention may be made of titanium dioxide, optionally with surface treatment, zirconium oxide or cerium oxide, and zinc oxide, iron oxide (black, yellow or red) or chromium oxide, manganese violet, ultramarine blue, chromium hydrate and ferric blue. Among organic pigments mention may be made of carbon black, organic lake pigments of barium, strontium, calcium or aluminium, including those certified by the U.S. Food and Drug Administration (FDA) (examples D&C or FD&C) and those exempt from FDA certification, such as lakes based on cochineal carmine. The pigments may range in amount from 0.1 to 50% in terms of active substance, for example, from 0.5 to 35%, such as from 2 to 25%, of the total weight of the composition.

The nacreous pigments may be chosen from white nacreous pigments such as titanium-covered mica and bismuth oxychloride; coloured nacreous pigments such as titanium mica with iron oxides, titanium mica with, for example, ferric blue or chromium oxide, titanium mica with an organic pigment of the aforementioned type; and also nacreous pigments based on bismuth oxychloride. They may range from 0 to 25% (in terms of active substance) of the total weight of the composition, for example, from 0.1 to 15% (if they are present). Use may therefore be made of pigments having goniochromatic properties and metal effect pigments, such as those described in the application filed under number FR 0 209 246, the content of which is hereby incorporated into the present application.

The fillers may be chosen from mineral, organic, lamellar, andspherical fillers. Mention may be made of talc, mica, silica, kaolin, Nylon® powders (Orgasol® from Atochem), poly-β-alanine powders and polyethylene powders, powders of polymers of tetrafluoroethylene (Teflon®), lauroyllysine, starch, boron nitride, hollow microspheres such as Expancel® (Nobel Industries), Polytrap® (Dow Corning) and silicone resin microbeads (Tospearls® from Toshiba, for example), precipitated calcium carbonate, magnesium carbonate and bicarbonate, hydroxyapatite, hollow silica microspheres (Silica Beads® from Maprecos), glass or ceramic microcapsules, metal soaps derived from organic carboxylic acids comprising from 8 to 22 carbon atoms, such as from 12 to 18 carbon atoms, for example zinc stearate, magnesium stearate or lithium stearate, zinc laurate and magnesium myristate.

Wax

Depending on the type of application envisaged, the composition according to the disclosure may further comprise at least one wax.

A wax for the purposes of the present disclosure is a lipophilic fatty compound which is solid at ambient temperature (25° C.) and exhibits a reversible solid/liquid state change, the compound having a melting temperature of greater than 40° C. and possibly up to 200° C. and having an anisotropic crystalline organization in the solid state. The size of the crystals is such that they diffract and/or scatter light, thereby endowing the composition with a turbid, more or less opaque appearance. By bringing the wax to its melting temperature it can be made miscible with the oils and made to form a microscopically homogeneous mixture; however, by bringing the temperature of the mixture back to ambient temperature, the wax is recrystallized in the oils of the mixture. It is this recrystallization in the mixture which is responsible for the decrease in the gloss of the mixture.

Linear hydrocarbon waxes may be used in the context of the present disclosure. Their melting point is advantageously greater than 35° C., for example greater than 55° C., such as greater than 80° C.

The linear hydrocarbon waxes are advantageously chosen from substituted linear alkanes, unsubstituted linear alkanes, unsubstituted linear alkenes, substituted linear alkenes, an unsubstituted compound being comprised solely of carbon and hydrogen, and wherein the substituents mentioned above do not contain any carbon atoms.

Linear hydrocarbon waxes include polymers and copolymers of ethylene with a molecular weight ranging from 400 to 800, for example Polywax 500 or Polywax 400 sold by New Phase Technologies.

Linear hydrocarbon waxes include linear paraffin waxes, such as the paraffins S&P 206, S&P 173 and S&P 434 from Strahl & Pitsch.

Linear hydrocarbon waxes include long-chain linear alcohols, such as the products comprising a mixture of polyethylene and alcohols comprising from 20 to 50 carbon atoms, for example, Performacol 425 or Performacol 550 (mixture in 20/80 proportions) sold by New Phase Technologies.

The waxes may be present in an amount ranging from 2 to 30% by weight in the composition, for example, from 5 to 20%, such as from 5 to 15%, so as not excessively to diminish the gloss of the composition and of the film deposited on the lips and/or skin.

Additives and pharmaceuticals

The composition of the disclosure may further comprise at least one cosmetic or dermatological active such as those conventionally employed.

As cosmetic, dermatological, hygienic or pharmaceutical actives which can be used in the composition disclosed herein mention may be made of moisturizers, vitamins, essential fatty acids, sphingolipids and sunscreens. These actives are used in an amount which is customary for the person skilled in the art, and, for example, at concentrations from 0 to 20%, such as from 0.001 to 15% of the total weight of the composition.

The composition may further comprise any other additive commonly used in such compositions, such as water, antioxidants, perfumes, preservatives and essential oils.

The person skilled in the art will of course take care to select this or these optional complementary compounds, and/or their amount, such that the advantageous properties of the composition according to the invention are not, or not substantially, adversely affected by the intended addition.

In one non-limiting embodiment of the disclosure the compositions according to the invention may be prepared in customary manner by the person skilled in the art. They may be present in the form of a cast product, and for example in the form of a stick or crayon, or in the form of a dish, which can be used by direct contact or with a sponge. For example, they find application as a cast foundation, cast blusher or cast eyeshadow, lipstick, a care base or care balm for the lips or a concealer product. They may also be present in the form of a soft paste or else a gel, a more or less fluid cream, or a liquid, which are applied by means of a tube or with the finger. They may therefore be composed of foundations, or lipsticks, sun products or skin colouring products.

In one embodiment, the composition of the invention is anhydrous and in that case comprises less than 5% of water relative to the total weight of the composition.

These compositions for topical application may, for example, comprise a cosmetic, dermatological, hygienic or pharmaceutical composition for the protection, treatment or care of the face, neck, hands or body (for example care cream, sun oil, body gel), a makeup composition (for example a makeup gel, cream or stick) or an artificial tanning composition or skin protection composition.

The composition disclosed herein may be in the form of a dermatological or care composition for the skin and/or epidermal derivatives or in the form of a sun protection composition or a body hygiene composition, for example, in the form of a deodorant. In that case it is present, for example, in an uncoloured form. It may therefore be used as a care base for the skin, epidermal derivatives or lips (lip balms, protecting the lips from the cold and/or sun and/or wind, care cream for the skin, nails or hair).

The compositions disclosed herein may be lipsticks in the form of a stick or in fluid form.

In one embodiment, the composition disclosed herein must of course be cosmetically or dermatologically acceptable; that is, it must comprise a non-toxic, physiologically acceptable medium which can be applied to the skin, epidermal derivatives or lips of the face of human beings. A cosmetically acceptable composition for the purposes of the disclosure is a composition whose appearance, odour and feel are pleasant.

Two-coat makeup product

The present disclosure likewise provides a cosmetic makeup product comprising first and second compositions, the first composition being composed of the composition described above, and the second composition comprising a second physiologically acceptable medium.

A makeup product is a product containing a colorant which allows a colour to be deposited on the keratin material (skin, lips or epidermal derivatives) of a human being by the application to the keratin material of products such as lipsticks, rouges, eyeliners, foundations or self-tanning products or semipermanent makeup products (tattoos).

When the makeup product is intended for making up the lips the first composition may be in the form of a stick or in a fluid form and the second composition may also be in the form of a stick or in a fluid form. A lip makeup product according to the disclosure comprises, for example, a first composition in the form of a stick, and a second composition in the form of a stick or in a fluid form packaged in a tube. The product disclosed herein may comprise two (or more) physiologically acceptable compositions packaged separately or together in a single container or in two (or more) separate or discrete containers.

In one non-limiting embodiment these compositions are packaged separately and, advantageously, in a single container having two ends.

The present disclosure therefore provides, for example, a cosmetic makeup product in the form of a foundation, blusher or eyeshadow, a lipstick, a product having care properties, for example, an eyeliner, a concealer product, or a body makeup product (of the tattoo type). The cosmetic makeup product is, for example, a lipstick.

The disclosure further provides a makeup kit comprising a cosmetic makeup product as defined above, in which the various compositions are packaged separately and are accompanied by appropriate means of application. These means may be fine brushes, coarse brushes, pens, pencils, felts, quills, sponges, tubes and/or foams. The composition can also be applied using the finger.

The first composition of the product disclosed herein can be applied to the keratin material to form a basecoat, over which the second composition is deposited to give a topcoat or finish coat. Under the basecoat it is possible to apply an undercoat, whose composition is the same as or different from that of the second coat.

It is also possible to apply to the first topcoat a second topcoat identical to or different from the first in composition. The resulting makeup is in one embodiment a makeup comprising a basecoat and a topcoat.

In some embodiments, the first composition is a foundation, face powder, lipstick, lipgloss, eyeliner or body makeup product and the second composition is a care product or a product intended for preserving or enhancing the cosmetic properties of the first composition, such as its gloss and comfort.

The disclosure also relates to a method of making up the skin and/or lips and/or epidermal derivatives, which comprises applying to the skin and/or lips and/or epidermal derivatives a cosmetic makeup product as defined above.

The disclosure further provides a method of making up the skin and/or lips and/or epidermal derivatives of a human being, which comprises applying to the skin, lips and/or epidermal derivatives a first coat of a first composition comprising, in a physiologically acceptable medium, polymer particles dispersed in a liquid organic phase and a colorant and then applying, over all or part of the first coat, a second coat of a second composition comprising a physiologically acceptable medium.

More specifically, in one embodiment, the method according to the disclosure comprises applying to the skin, lips and/or epidermal derivatives of a human being a first coat of a first composition comprising, in a first physiologically acceptable medium, polymer particles dispersed in a liquid organic phase and a colorant, in leaving the first coat to dry, and then applying, over all or part of the first coat, a second coat of a second composition comprising a second physiologically acceptable medium.

This two-coat makeup may, in one embodiment, be adapted to all makeup products for the skin both of the face and of the body, the mucosae, such as the lips, the inside of the lower eyelids, and the epidermal derivatives, such as nails, eyelashes, hair, eyebrows or even body hairs. The second coat can form designs and can be applied with a pen, pencil or any other instrument (sponge, finger, fine brush, coarse brush, quill, etc.). This makeup may also be applied to makeup accessories, such as false nails, false eyelashes, wigs or else discs or patches adhering to the skin or lips (of the beauty-spot type).

The disclosure additionally provides a made-up substrate comprising a first coat of a first composition comprising, in a first physiologically acceptable medium, polymer particles dispersed in a liquid phase, and a second coat of a second composition which is deposited over all or part of the first coat and comprises a second physiologically acceptable medium.

Second composition

In one aspect of the disclosure the second composition according to the invention comprises at least one apolar or relatively apolar compound, which may be chosen from oils, gums and waxes. The second composition comprises, for example, more than 70%, for further example, more than 80% by weight, such as 100% by weight of apolar or relatively apolar compounds. These apolar or relatively apolar compounds are, for example, silicone compounds, colorants or gelling agents.

The second composition may be, for example, one of the compositions described in application U.S. 60/375 814, the content of which is hereby incorporated into the present application.

In one non-limiting embodiment of the disclosure the physiologically acceptable medium of the second composition comprises a liquid phase which is non-volatile at ambient temperature and atmospheric pressure.

Non-volatile liquid phase of the second composition

The second composition may comprise from 15 to 90% by weight of at least one non-volatile liquid phase.

A “non-volatile liquid phase” is any medium apt to remain on the skin or lips for a number of hours. A non-volatile liquid phase has, for example, a non-zero vapour pressure at ambient temperature and atmospheric pressure, of less than 0.02 mm Hg (2.66 Pa), for example, less than 10⁻³ mm Hg (0.13 Pa).

The non-volatile liquid phase of the second composition may be, for example, apolar or relatively apolar and, for example, comprises at least one of a liquid hydrocarbon oil, silicone oil and fluoro oil.

The non-volatile oils may be chosen from polydimethylsiloxanes; alkyldimethicones; polyphenylmethylsiloxanes such as phenyldimethicones and phenyltrimethicones; and silicones modified with aliphatic and/or aromatic groups, optionally fluorinated, or with functional groups such as hydroxyl, thiol and amine groups.

One non-volatile oil is an oil selected from the silicones of formula (I):

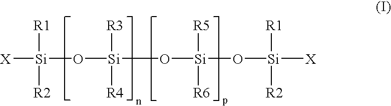

wherein:

R₁, R₂, R₅ and R₆, which may be identical or different, are chosen fromalkyl radicals comprising from 1 to 6 carbon atoms,

R₃ and R₄, which may be identical or different, are chosen from alkyl radicals comprising from 1 to 6 carbon atoms and aryl radicals,

X is an alkyl radical comprising from 1 to 6 carbon atoms, a hydroxyl radical or a vinyl radical, and

n and p are selected so as to endow the oil with a weight-average molecular mass of less than 200 000 g/mol, for example, less than 150 000 g/mol, such as less than 100 000 g/mol.

The non-volatile silicone oil of formula (I) may be, for example, a polydimethylsiloxane with a viscosity ranging from 0.5 to 60 000 cSt, for example, from 0.5 to 10,000 cSt, for further example, from 0.5 to 1,000 cSt, such as DC 200, with a viscosity of 350 cSt, which is sold by Dow Corning.

In one non-limiting embodiment the non-volatile silicone oil of formula (I) is a polydimethylsiloxane with a viscosity ranging from 0.5 to 500 cSt, such as from 1 to 10 cSt.

The non-volatile liquid phase of the second composition may comprise, for example, at least one fluoro oil of formula (II):

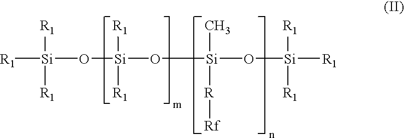

wherein:

R is chosen from linear and branched alkylenyl groups comprising from 1 to 6 carbon atoms, such as a divalent methylenyl, ethylenyl, propylenyl or butylenyl group,

Rf is a fluoroalkyl radical, for example, a perfluoroalkyl radical, comprising from 1 to 9 carbon atoms, such as from 1 to 4 carbon atoms,

R₁ is, independently at each occurrence, a C₁-C₂₀ alkyl radical, for example, a C₁-C₄ alkyl radical, a hydroxyl, or phenyl radical,

m ranges from 0 to 150, such as from 20 to 100, and

n is from 1 to 300, such as from 1 to 100.

Oils of formula (II) may be used such that R₁ is a methyl, R is an ethyl and Rf is CF₃. As fluorosilicone compounds of formula (II) mention may be made, for example, of those sold by Shin Etsu under the names X22-819, X22-820, X22-821 and X22-822 or else FL-100.

Among the fluoro oils mention may also be made of fluorinated polyethers chosen from the compounds of formula (III):

R₆—(CF₂—CFR₃—CF₂O)_{p—}(CFR_{4—}CF₂—O)_{q}—(CFR₅—O)ᵣ—R₇ (III)

wherein:

R₃ to R₆, which may be identical or different, are monovalent radicals chosen from —F,

(CF₂)n—CF₃ and —O—(CF₂)ₙ—CF₃,

R₇ is a monovalent radical chosen from —F and —(CF₂)ₙ—CF₃,

n ranges from 0 to 4 inclusive,

p ranges from 0 to 600, q ranges from 0 to 860, r ranges from 0 to 1,500, p, q and r being integers selected such that the weight-average molecular mass of the compound is from 500 to 100 000, such as from 500 to 10,000.

The fluoro oils may also be chosen from fluorinated alkanes, for example, C2-C50 fluorinated alkanes, for further example, C5-C30, fluoroalkanes and perfluoroalkanes, such as perfluorodecalin, perfluoroadamantane and bromoperfluorooctyl and mixtures thereof.

The second composition may comprise a high molecular weight silicone polymer.

When the second composition disclosed herein is liquid it may comprise 20 to 50% by weight of a high molecular weight silicone polymer.

When the second composition disclosed herein is solid it may comprise from 2 to 40% by weight of a high molecular weight silicone polymer.

This polymer may be liquid or solid at ambient temperature and its weight-average molecular mass is greater than or equal to 200,000 g/mol, for example, from 200,000 to 2,500,000, such as from 200 000 to 2,000,000 g/mol.

The viscosity of this silicone polymer may be between 10,000 and 5,000,000 cSt, for example, from 100,000 to 1,000,000 cSt, such as from 300,000 to 700,000 cSt, measured in accordance with standard ASTM D-445.

The high molecular weight silicone polymer may be an ungrafted polymer, i.e. a polymer obtained by polymerizing at least one monomer without subsequent reaction of the side chains with another chemical compound. The silicone polymer may be chosen from, for example, dimethiconols, fluorosilicones, dimethicones and mixtures thereof. The polymer may be a homopolymer.

Use may be made in particular of a high molecular weight silicone polymer corresponding to the formula (IV):

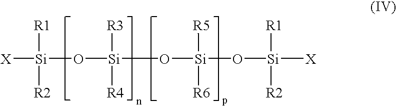

wherein:

R₁, R₂, R₅ and R₆, which may be identical or different, are chosen from alkyl radicals comprising from 1 to 6 carbon atoms optionally substituted by at least one fluorine atom,

R₃ and R₄, which may be identical or different, are chosen from alkyl radicals comprising from 1 to 6 carbon atoms and aryl radicals,

X is an alkyl radical comprising from 1 to 6 carbon atoms, a hydroxyl radical, a vinyl or allyl radical or an alkoxy radical comprising from 1 to 6 carbon atoms, and

n and p are selected such that the silicone compound has a weight-average molecular mass of greater than or equal to 200,000 g/mol.

p may be, for example, 0.

The polymers of formula (IV) where R₁ to R₆ represent a methyl group and the substituent X represents a hydroxyl group are dimethiconols. Examples are the polymers of formula (III) where p=0 and n ranges from 2,000 and 40,000, for example, from 3,000 to 30,000.

The polymers have, for example, a molecular mass ranging from 1,500,000 to 2,000,000 g/mol.

In one non-limiting embodiment the high molecular weight silicone polymer is the dimethiconol sold by Dow Corning in a polydimethylsiloxane (5 cSt) under the reference D2-9085, the viscosity of the mixture being 1,550 cSt, or the dimethiconal sold by Dow Corning in octamethylcyclotetrasiloxane under the reference DC 1503. Likewise dimethiconol (with a molecular weight of 1 770 000 g/mol) sold by Dow Corning under the reference Q2-1403 or Q2-1401, may be used, the viscosity of the mixture being 4,000 cSt.

As high molecular weight silicone polymers of formula (IV) which can be used according to the disclosure mention may be made of those for which:

the substituents R₁ to R₆ and X are a methyl group, such as that sold under the name SE30 by General Electric and that sold under the name AK 500000 by Wacker,

the substituents R₁ to R₆ and X are a methyl group and p and n are such that the molecular weight is 250,000 g/mol, such as that sold under the name Silbione 70047 V by Rhodia,

the substituents R₁ to R₆ are a methyl group and the substituent X is a hydroxyl group, such as that sold under the name Q2-1401 or Q2-1403 by Dow Corning,

the substituents R₁, R₂, R₅, R₆ and X are a methyl group, the substituents R₃ and R₄ are an aryl group and n and p are such that the molecular weight of the polymer is 600,000 g/mol, such as that sold under the name 761 by Rhône-Poulenc.

The high molecular weight silicone polymer may be introduced into the composition in the form of a mixture with a liquid silicone, the viscosity of the liquid silicone being between 0.5 and 10,000 cSt, for example, from 0.5 to 500 cSt, such as from 1 to 10 cSt.

The silicone fluid may be chosen from polyalkylsiloxanes, polyarylsiloxanes, polyalkylarylsiloxanes and mixtures thereof. The liquid silicone may be a volatile silicone such as a cyclic polydimethylsiloxane comprising from 3 to 7 —(CH₃)₂SiO— units.

The liquid silicone may also be a non-volatile polydimethylsiloxane silicone, for example, with a viscosity of between 0.5 and 10,000 cSt, for example, of the order of 5 cSt, an example being the silicone sold as DC 200 by Dow Corning.

The proportion of the high molecular weight silicone polymer in the high molecular weight silicone polymer/liquid silicone mixture may be, for example, 10/90 and 20/80. The viscosity of the high molecular weight silicone polymer/liquid silicone mixture may be, for example, from 1,000 and 10,000 cSt.

The high molecular weight dimethicones according to the disclosure include the dimethicones described in U.S. Pat. No. 4,152,416. They are sold, for example, as SE30, SE33, SE54 and SE76.

The dimethicones according to the disclosure are, for example, compounds of formula (IV) such that R₁ to R₆ and X are methyls and p=0. The molecular weight of these polymers may range, for example, from 200,000 to 300,000, such as from 240,000 to 260 000 g/mol.

The dimethicones according to the invention include polydimethylsiloxanes, (polydimethylsiloxane)(methylvinylsiloxane) copolymers, poly(dimethylsiloxane)(diphenyl)(methylvinylsiloxane) copolymers, and mixtures thereof.

The high molecular weight fluorosilicones according to the disclosure may have, for example, a molecular weight ranging from 200,000 to 300,000, such as from 240,000 to 260,000 g/mol.

Wax in the second composition

The second composition may also comprises at least one wax, for example when it is in solid form.

The waxes may be present in an amount ranging from 2 to 30% by weight in the composition, for example, from 5 to 20%, such as from5 to 15%, of the composition.

Linear hydrocarbon waxes may be used in the context of the present disclosure. Their melting point may be greater than 35° C., for example greater than 55° C., such as greater than 80° C.

The linear hydrocarbon waxes may be chosen from substituted linear alkanes, unsubstituted linear alkanes, unsubstituted linear alkenes, substituted linear alkenes, an unsubstituted compound being comprised solely of carbon and hydrogen, and wherein the substituents mentioned above do not contain carbon atoms.

Linear hydrocarbon waxes include polymers and copolymers of ethylene with a molecular weight ranging from 400 to 800, for example Polywax 500 or Polywax 400, sold by New Phase Technologies.

Linear hydrocarbon waxes include linear paraffin waxes, such as paraffins S&P 206, S&P 173 and S&P 434 from Strahl & Pitsch.

Linear hydrocarbon waxes include long-chain linear alcohols, such as the products comprising a mixture of polyethylenes and alcohols comprising from 20 to 50 carbon atoms, in particular Performacol 425 or Performacol 550 (mixture in 20/80 proportions) sold by New Phase Technologies.

The second composition may also comprise a silicone wax, such as a dimethicone comprising alkyl groups at the chain end. These alkyl groups, for example, may have more than 18 carbon atoms, for further example from 20 to 50, such as from 30 to 45 carbon atoms.

The silicone wax corresponds for example to the formula (V) or (VI)

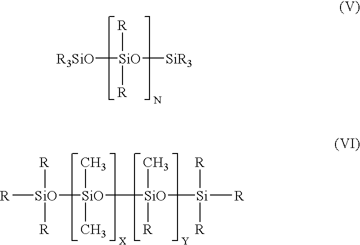

wherein R is an alkyl, X is greater than or equal to zero, and N and Y are greater than or equal to one.

R comprises from 1 to 50 carbon atoms, subject to the proviso that the compound is solid at ambient temperature.

Examples of silicone waxes include:

C20-C24 alkyl methicones, C24-C28 alkyl dimethicones, C20-C24 alkyl dimethicones and C24-C28 alkyl dimethicones, sold by Archimica Fine Chemicals as SilCare 41 M40, SilCare 41 M50, SilCare 41 M70 and SilCare 41 M80,

stearyl dimethicones referenced SilCare 41 M65, sold by Archimica, or referenced DC-2503, sold by Dow Corning,

stearoxytrimethylsilanes sold as SilCare 1 M71 or DC-580

the products Abil Wax 9810, 9800 or 2440 from Wacker-Chemie GmbH,

C30-45 alkyl methicones sold by Dow Corning as AMS-C30 Wax, and also C30-45 alkyl dimethicones sold as SF1642 or SF-1632 by General Electric.

Gelling agent

The second composition may comprise a gelling agent as described herein.

Colorant

The second composition of the cosmetic makeup product according to the disclosure may comprise a colorant, which may be chosen from water-soluble and fat-soluble dyes, pigments, nacres and mixtures thereof.

Pigments are white or coloured, organic or mineral particles which are insoluble in the liquid organic phase and are intended for colouring and/or opacifying the first composition.

Nacres are iridescent particles, produced in particular by certain molluscs within their shell, or else synthesized, which are insoluble in the medium of the first composition.

Dyes are compounds, generally organic compounds, which are soluble in fatty substances, such as oils, or in an aqueous-alcoholic phase.

The fat-soluble dyes are, for example, Sudan Red, D&C Red No.17, D&C Green No. 6, β-carotene, soya oil, Sudan Brown, D&C Yellow No. 11, D&C Violet No. 2, D&C Orange No. 5, quinoline yellow, annatto and bromo acids.

The water-soluble dyes are, for example, beetroot juice, methylene blue and caramel.

The pigments may be white or coloured pigments, mineral and/or organic pigments, interference pigments or non-interference pigments. Among mineral pigments mention may be made of titanium dioxide, optionally with surface treatment, zirconium oxide or cerium oxide, and zinc oxide, iron oxide (black, yellow, brown or red) or chromium oxide, manganese violet, ultramarine blue, chromium hydrate and ferric blue. Among organic pigments mention may be made of carbon black, organic lake pigments of barium, strontium, calcium or aluminium, including those certified by the U.S. Food and Drug Administration (FDA) (examples D&C or FD&C) and those exempt from FDA certification, such as lakes based on cochineal carmine.

The nacreous pigments may be chosen from white nacreous pigments such as titanium-covered mica and bismuth oxychloride, coloured nacreous pigments such as titanium mica with iron oxides, titanium mica with, for example, ferric blue or chromium oxide, titanium mica with an organic pigment of the aforementioned type, and also nacreous pigments based on bismuth oxychloride. Use may therefore be made of pigments having goniochromatic properties, for example, with liquid crystals or multilayered, and/or metal effect pigments, such as those described in the application filed under number FR 0 209 246, the content of which is hereby incorporated into the present application.

The second composition of the product according to the disclosure may also, but need not necessarily, comprise a colorant.

Generally speaking, the colorants range in amount from 0.001 to 60%, for example, from 0.01 to 50%, such as from 0.1 to 40% of the total weight of each first and second composition.

The colorant or filler may additionally be present in the form of a “particle paste”.

Pharmaceuticals of the makeup product

Each composition of the two-coat makeup product according to the disclosure may be present in any pharmaceutical form which is normally used for topical application, for example, in the form of an oily or aqueous solution, an oily or aqueous gel, an oil-in-water or water-in-oil emulsion, a multiple emulsion, a dispersion of oil in water which is mediated by vesicles, the vesicles being situated at the oil/water interface, or a powder. Each composition may be fluid or solid.

The first or the second composition, or both, may have a continuous fatty phase and are, for example, in anhydrous form, and may comprise less than 5% of water, for example less than 1% of water, relative to the total weight of the first or second composition. For example, the overall two-coat makeup product may be in anhydrous form.

Each first and second composition may have the appearance of a lotion, cream, ointment, soft paste, salve, a cast or moulded solid, for example, in stick form or dish form, or else a compacted solid.

Each composition may be, for example, in the form of a more or less rigid stick.

Each composition may be packaged separately in a single container, for example in a twin-compartment pen, the base composition being delivered by one end of the pen and the top composition being delivered by the other end of the pen, each end being closed, for example, tightly, by means of a cap.

The composition which is applied in a first coat is, for example in solid form, allowing more practical application, better temporal stability and temperature stability of the composition, and a precise line of the makeup, which is highly desirable in the case of a lipstick or an eyeliner.

The product according to the disclosure may be used for making up the skin and/or lips and/or epidermal derivatives, in accordance with the nature of the ingredients employed. For example, the product of the disclosure may be in the form of a solid foundation, a lipstick or lipstick paste, a concealer product, an eyeliner, a mascara, an eyeshadow, a body makeup product or else a skin-colouring product.

The product is, for example, a lipstick.

The first and/or the second composition may be in solid form.

The topcoat, in one embodiment has properties of care, gloss and transparency.

The invention further provides a lip product, a foundation, a tattoo, a blusher or an eyeshadow which comprises a first and a second composition as described above.

The compositions of the disclosure may be obtained by heating the various constituents at the melting temperature of the highest waxes, then casting the melted mixture in a mould (dish or glove finger). They may also be obtained by extrusion as described in application EP-A-0 667 146.

The invention is illustrated in more detail in the following examples. The percentages are percentages by weight.

### EXAMPLE 1

### Polymer Dispersion

A dispersion of an uncrosslinked copolymer of methyl acrylate and acrylic acid in a 95/5 ratio in isododecane by the method of Example 1 of EP-A-749 746, replacing the heptane by isododecane, was prepared. This gave a dispersion of poly(methyl acrylate/acrylic acid) particles surface-stabilized in isododecane by a block polystyrene/copoly(ethylene-propylene) diblock copolymer sold under the name Kraton G1701, having a solids content of 25% by weight.

### EXAMPLE 2

### Lipstick

| | |
|---|---|
| Polyethylene wax | 10.5% |
| (weight-average molecular mass: 500) | |
| Linear fatty alcohols | 2.5% |
| (Performacol 550 alcohol, sold by | |
| New Phase Technologies) | |
| Dispersion of Example 1 | 68% |
| Sucrose acetate isobutyrate | 5% |
| (Eastman SAIB sold by Eastman Chemical) | |
| Pigment paste | 13.5% |
| Perfume | 0.5% |

A heating vessel was charged with the polyethylene wax, the C30-C50 alcohols and the pigment paste, which were heated at 100° C. with magnetic stirring to give a homogeneous mixture. The composition of the pigment paste was 70% pigments, 1% poly(12-hydroxystearic) stearate and 29% hydrogenated polyisobutene. Subsequently the dispersion of Example 1 and the sucrose acetate isobutyrate were added, while the temperature and the stirring were maintained. The composition was cast into moulds at 40° C. and the moulds were cooled at −20° C. for thirty minutes. The sticks were then demoulded. The resulting sticks were homogeneous in colour and went on well. The sticks provided a deposit on the lips which did not have staying power, did not migrate or transfer and was not sticky.

### EXAMPLE 3

### Lipstick

| | |
|---|---|
| Polystyrene/poly(ethylene-propylene) | 0.5% |
| Kraton G-1650E | |
| (sold by Kraton) | |
| Dispersion of Example 1 | 65% |
| Polycaprolactone | 10% |
| (Capa 2125, manufactured by Solvay) | |
| Sucrose acetate isobutyrate | 10% |
| (Eastman SAIB, sold by Eastman Chemical) | |
| Pigment paste | 13.5% |
| Fragrance | qs |
| Preservative | qs |

The polycaprolactone was melted at 100° C. and then the pigment paste, whose composition was identical to that of Example 2, was added with stirring. Subsequently the other ingredients were added until a homogeneous mixture was obtained. The mixture was subsequently cooled to ambient temperature and packaged in small pots.

### EXAMPLE 4

### Liquid second composition

| | |
|---|---|
| Polydimethylsiloxane sold | 40% |
| as Silbione 70047 V by Rhodia | |
| (500 000 cSt-250 000 g/mol) | |
| Polydimethylsiloxane sold | 60% |
| by Dow Corning as DC200 (5 cSt) | |

The two ingredients were mixed at 70° C. using a Rayneri stirrer.

### EXAMPLE 5

### Solid second composition

| | % by weight |
|---|---|
| Silicone oil (PDMS) | 25 |
| DC200 from Dow Corning (5 cSt) | |
| Dimethicone (and) dimethiconol | |
| D2-9085 from Dow Corning (1550 cSt) | 61 |
| Trifluoropropyl dimethicone (100 cSt) | |
| X22-819 from Shin Etsu | 1 |
| C30-45 alkyl dimethicone | 5 |
| (SF 1642 from GE Bayer Silicone) | |
| Polyethylene wax (weight-average | 8 |
| MW: 500) | |

The silicone oil, dimethiconol and fluorinated dimethicone were mixed in hot form until a homogeneous mixture was formed. Thereafter the C₃₀-C₄₅ alkyl dimethicone was added to the first mixture, which was heated at 110° C. The polyethylene wax was subsequently added gradually until a homogeneous mixture was obtained. The mixture was cooled to 90-95° C. and then poured into moulds, which were cooled at −20° C. for thirty minutes. The sticks were then demoulded.

What is claimed is:

1. A composition comprising
a) at least one liquid fatty phase,
b) a dispersion of at least one polymer particle dispersed in the at least one liquid fatty phase, and
c) at least one compound chosen from esters of aliphatic and aromatic polycarboxylic acids and aliphatic and aromatic alcohols comprising from 1 to 10 carbon atoms, the at least one compound being present in an amount sufficient to plasticize the at least one polymer particle.

2. The composition according to claim 1, wherein the aliphatic and aromatic polycarboxylic acids comprise from 3 to 12 carbon atoms.

3. The composition according to claim 2, wherein the aliphatic and aromatic polycarboxylic acids comprise from 3 to 10 carbon atoms.

4. The composition according to claim 3, wherein the aliphatic and aromatic polycarboxylic acids comprise from 6 to 8 carbon atoms.

5. The composition according to claim 1, wherein the aliphatic and aromatic alcohols comprise from 1 to 10 carbon atoms, and may be chosen from alcohols of the forumula R1OH
wherein R1 is a radical chosen from methyl, ethyl, propyl, isopropyl, butyl, hexyl, ethylhexyl, decyl, isodecyl and benzyl substituted with a C₁-C₄ alkyl group.

6. The composition according to claim 5, wherein the aliphatic and aromatic alcohols comprise from 1 to 8 carbon atoms.

7. The composition according to claim 6, wherein the aliphatic and aromatic alcohols comprise from 1 to 4 carbon atoms.

8. The composition according to claim 1, wherein the aliphatic and aromatic polycarboxylic acids are chosen from dicarboxylic acids corresponding to the formula HOOC—(CH₂)ₙ—COOH, where n ranges from 1 to 10.

9. The composition according to claim 8, where n ranges from 2 to 8.

10. The composition according to claim 8, wherein the dicarboxylic acids are chosen from succinic, adipic and sebacic acid.

11. The composition according to the claim 10, wherein the at least one compound is chosen from diethylhexyl succinate and diethyl succinate.

12. The composition according to claim 10, wherein the at least one compound is not diisopropyl adipate.

13. The composition according to claim 10, wherein the at least one compound is chosen from dibutyl adipate and di-2-ethylhexyl adipate.

14. The composition according to claim 10, wherein the at least one compound is chosen from dibutyl sebacate, diethylhexyl sebacate, diethyl sebacate and diisopropyl sebacate.

15. The composition according to claim 1, wherein the aliphatic and aromatic polycarboxylic acids are chosen from phthalic acid and its derivatives,

16. The composition according to claim 15, wherein the aliphatic and aromatic polycarboxylic acids are chosen from butyl benzyl phthalate, dibutyl phthalate, diethylhexyl phthalate, diethyl phthalate and dimethyl phthalate.

17. The composition according to claim 1, wherein the aliphatic and aromatic polycarboxylic acids are chosen from tricarboxylic acids corresponding to the formula 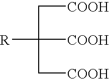
wherein R is a hydrocarbon radical.

18. The composition according to claim 17, wherein R is chosen from H, —OH and —OCOR′ wherein R′ is an alkyl group.

19. The composition according to claim 18, wherein R′ is chosen from an alkyl group comprising from 1 to 6 carbon atoms.

20. The composition according to claim 17, wherein the tricarboxylic acids are chosen from acetylcitric acid and its derivatives.

21. The composition according to claim 20, wherein the at least one compound is not tributyl acetylcitrate.

22. The composition according to claim 12, wherein the at least one compound is chosen from triethyl acetylcitrate, triethylhexyl acetylcitrate, trihexyl acetylcitrate, trihexyl butyroylcitrate, isodecyl citrate, isopropyl citrate, tributyl citrate and triethylhexyl citrate.

23. The composition according to claim 1, wherein the at least one compound comprises no polar group chosen from ionic and non-ionic polar groups

24. The composition according to claim 23, wherein the polar group is chosen from —COOH; —OH; ethylene oxide; propylene oxide; —PO₄; —NHR; —NR₂R₃, wherein R₂ and R₃, which may be identical or different, are chosen from linear and branched C₁ to C₂₀ alkyl and alkoxy radicals.

25. The composition according to claim 1, wherein the at least one compound has a solubility parameter, δₕ, ranging from 5.5 to 11 (J/cm³)^{1/2}.

26. The composition according to claim 25, wherein the at least one compound has a solubility parameter, δₕ, ranging from 5.9 to 11 (J/cm³)^{1/2}.

27. The composition according to claim 26, wherein the at least one compound has a solubility parameter, δₕ, ranging from 7 to 10.5 (J/cm³)^{1/2}.

28. The composition according to claim 27, wherein the at least one compound has a solubility parameter, δₕ, ranging from 8 to 10 (J/cm³)^{1/2}.

29. The composition according to claim 28, wherein the at least one compound has a solubility parameter, δₕ, ranging from 9 to 10 (J/cm³)^{1/2}.

30. The composition according to claim 1, wherein the at least one compound has a solubility parameter, δₚ, ranging from 1.5 to 4.5 (J/cm³)^{1/2}.

31. The composition according to claim 30, wherein the at least one compound has a solubility parameter, δₚ, ranging from 1.5 to 3.5 (J/cm³)^{1/2}.

32. The composition according to claim 31, wherein the at least one compound has a solubility parameter, δₚ, ranging from 2 to 3 (J/cm³)^{1/2}.

33. The composition according to claim 1, wherein the at least one compound is present in an amount ranging from 0.1 and 25% by weight, relative to the total weight of the composition.

34. The composition according to claim 33, wherein the at least one compound is present in an amount ranging from 0.5 to 15% by weight, relative to the total weight of the composition.

35. The composition according to claim 34, wherein the at least one compound is present in an amount ranging from 3 to 10% by weight, relative to the total weight of the composition.

36. The composition according to claim 1, wherein the mass ratio between the at least one polymer particle and the at least one compound ranges from 0.5 to 100.

37. The composition according to claim 36, wherein the mass ratio between the at least one polymer particle and the at least one compound ranges from 1 to 50.

38. The composition according to claim 37, wherein the mass ratio between the at least one polymer particle and the at least one compound ranges from 1 to 10.

39. The composition according to claim 38, wherein the mass ratio between the at least one polymer particle and the at least one compound ranges from 1 to 5.

40. The composition according to claim 1, wherein the at least one polymer particle is insoluble in water-soluble alcohols.

41. The composition according to claim 1, wherein the at least one polymer particle is chosen from polyurethanes, acrylic polyurethanes, polyureas, polyurea-polyurethanes, polyester-polyurethanes, polyether-polyurethanes, polyesters, polyester amides, fatty-chain polyesters, alkyds, acrylic polymers and copolymers, vinyl polymers and copolymers, acrylic-silicone copolymers; polyacrylamides, silicone polymers, and fluoro polymers.

42. The composition according to claim 1, wherein the at least one polymer particle is filmable.

43. The composition according to claim 1, wherein the at least one polymer particle dispersed in the at least one liquid fatty phase ranges from 5 to 40% by weight of the dry-matter content of the composition.

44. The composition according to claim 43, wherein the at least one polymer particle dispersed in the at least one liquid fatty phase ranges from 5 to 35% by weight of the dry-matter content of the composition.

45. The composition according to claim 44, wherein the at least one polymer particle dispersed in the at least one liquid fatty phase ranges from 8 to 30% by weight of the dry-matter content of the composition.

46. The composition according to claim 1, wherein the at least one polymer particle is surface-stabilized in the at least one liquid fatty phase by a stabilizer chosen from block polymers, graft polymers, random polymers and mixtures thereof.

47. The composition according to claim 42, wherein the stabilizer is chosen from graft block polymers comprising at least one block resulting from the polymerization of diene and at least one block of a vinyl polymer and block polymers comprising at least one block resulting from the polymerization of diene and at least one block of a vinyl polymer.

48. The composition according to claim 47, wherein the stabilizer is a diblock polymer.

49. The composition according to claim 1, wherein the composition further comprises a colloidal dispersion of at least one particle which is a solid at ambient temperature and is chosen from pigments and nacres.

50. The composition according to claim 49, wherein the colloidal dispersion ranges from 0.5 to 60% by weight, relative to the total weight of the composition.

51. The composition according to claim 50, wherein the colloidal dispersion ranges from 2 to 40% by weight, relative to the total weight of the composition.

52. The composition according to claim 51, wherein the colloidal dispersion ranges from 2 to 50% by weight, relative to the total weight of the composition.

53. The composition according to claim 49, wherein the colloidal dispersion comprises at least one particle dispersant chosen from poly(12-hydroxystearic) stearate, poly(12-hydroxystearic) acid, and diglyceryl 2-dipolyhydroxystearate.

54. The composition according to claim 49, wherein the colloidal dispersion comprises a fatty substance which is liquid at ambient temperature.

55. The composition according to claim 54, wherein the fatty substance which is liquid at ambient temperature is hydrogenated polyisobutene.

56. The composition according to claim 1, wherein the at least one liquid fatty phase comprises a non-volatile fatty phase and a volatile fatty phase.

57. The composition according to claim 56, wherein the volatile fatty phase comprises at least one oil chosen from C₈-C₁₆ isoalkanes.

58. The composition according to the claim 57, wherein the volatile fatty phase comprises at least one oil chosen from isododecane and isohexadecane.

59. The composition according to claim 1, wherein the composition is substantially free of silicone oil.

60. The composition according to claim 1, wherein the composition is substantially free of fatty alcohol.

61. The composition according to claim 56, wherein the non-volatile fatty phase is apolar.

62. The composition according to claim 57, wherein the non-volatile fatty phase comprises a hydrocarbon oil

63. The composition according to claim 62, wherein the hydrocarbon oil is hydrogenated polyisobutene.

64. The composition according to claim 1, wherein the composition further comprises a gelling agent chosen from polymeric gelling agents and mineral gelling agents.

65. The composition according to the claim 64, wherein the gelling agent is a polymeric gelling agent chosen from amorphous block copolymers of styrene and olefin.

66. The composition according to claim 65, wherein the polymeric gelling agent is a triblock copolymer chosen from styrene-ethylene/propylene-styrene copolymers, styrene-ethylene/butadiene-styrene copolymers, styrene-isoprene-styrene copolymers and styrene-butadiene-styrene copolymers.

67. The composition according to claim 66, wherein the triblock copolymer is hydrogenated.

68. The composition according to claim 64, wherein the gelling agent is present in an amount ranging from 0.1 to 5% by weight, relative to the total weight of the composition.

69. The composition according to claim 68, wherein the gelling agent is present in an amount ranging from 0.2 to 3% by weight, relative to the total weight of the composition.

70. The composition according to claim 69, wherein the gelling agent is present in an amount ranging from 0.5 to 2% by weight, relative to the total weight of the composition.

71. The composition according to claim 64, wherein the gelling agent is a polymeric gelling agent chosen from polycaprolactones.

72. The composition according to the claim 71, wherein the polycaprolactones are chosen from ε-caprolactone homopolymers with a molecular weight ranging from 300 to 2,000 g/mol.

73. The composition according to claim 1, wherein the composition further comprises at least one wax chosen from ethylene polymers and copolymers, and linear alcohols comprising from 20 to 50 carbon atoms.

74. The composition according to claim 1, wherein the composition is in a form chosen from a stick, a bar, a smooth paste, and a liquid.

75. The composition according to claim 1, wherein the composition is in anhydrous form.

76. The composition according to claim 1, wherein the composition is in the form of a product for caring for and/or making up the skin and/or lips.

77. The composition according to claim 1, wherein the composition is in a form chosen from a foundation, a blusher, an or eyeshadow, a lipstick, a care base for the lips, a care balm for the lips, a concealer product, an eyeliner and amascara.

78. A composition comprising
a) at least one liquid fatty phase,
b) a dispersion of at least one polymer particle dispersed in the at least one liquid fatty phase, and
c) at least one compound having a solubility parameter, δₕ, ranging from 5.5 to 11 (J/cm³)^{1/2}, the at least one compound being present in an amount sufficient to plasticize the at least one polymer particle.

79. The composition according to the claim 78, wherein the solubility parameter, δₕ, of the at least one compound ranges from 5.9 to 11 (J/cm³)^{1/2}.

80. The composition according to the claim 79, wherein the solubility parameter, δₕ, of the at least one compound ranges from 7 to 10.5 (J/cm³)^{1/2}.

81. The composition according to the claim 80, wherein the solubility parameter, δₕ, of the at least one compound ranges from 8 to 10 (J/cm³)^{1/2}.

82. The composition according to the claim 81, wherein the solubility parameter, δₕ, of the at least one compound ranges from 9 to 10 (J/cm³)^{1/2}.

83. The composition according to claim 78, wherein the at least one compound has a molecular mass of less than 5,000 g/mol.

84. The composition according to claim 83, wherein the at least one compound has a molecular mass of less than 2,000 g/mol.

85. The composition according to claim 84, wherein the at least one compound has a molecular mass of less than 1,000 g/mol.

86. The composition according to claim 85, wherein the at least one compound has a molecular mass of less than 900 g/mol.

87. The composition according to claim 78, wherein the at least one compound comprises no polar group chosen from ionic or non-ionic polar groups.

88. The composition according to claim 87, wherein the non-ionic polar groups are chosen from COOH; —OH; ethylene oxide; propylene oxide; —PO₄; —NHR; and —NR₁R₂, wherein R₁ and R₂, which may be identical or different, are chosen from linear and branched C₁ to C₂₀ alkyls and alkoxy radicals and optionally form a ring.

89. The composition according claim 78, wherein the solubility parameter, δₚ, of the compound ranges from 1.5 to 4.5 (J/cm³)^{1/2}.

90. The composition according to claim 88, wherein the solubility parameter, δₚ, of the compound ranges from 1.5 to 4 (J/cm³)^{1/2}.

91. The composition according to claim 89, wherein the solubility parameter, δₚ, of the compound ranges from 1.5 to 3.5 (J/cm³)^{1/2}.

92. The composition according to claim 90, wherein the solubility parameter, δₚ, of the compound ranges from 2 to 3 (J/cm³)^{1/2}.

93. The composition according to claim 78, wherein the compound is present in an amount ranging from 0.1 and 25% by weight, relative to the total weight of the composition.

94. The composition according to claim 78, wherein the compound is present in an amount ranging from 0.5 to 15% by weight, relative to the total weight of the composition.

95. The composition according to claim 78, wherein the compound is present in an amount ranging from 3 to 10% by weight, relative to the total weight of the composition.

96. The composition according to claim 78, wherein the mass ratio between the at least one polymer particle and the at least one compound ranges from 0.5 to 100.

97. The composition according to claim 95, wherein the mass ratio between the at least one polymer particle and the at least one compound ranges from 1 to 50.

98. The composition according to claim 96, wherein the mass ratio between the at least one polymer particle and the at least one compound ranges from 0.5 to 100.

99. The composition according to claim 97, wherein the mass ratio between the at least one polymer particle and the at least one compound ranges from 1 to 10.

100. The composition according to claim 98, wherein the mass ratio between the at least one polymer particle and the at least one compound ranges from 1 to 5.

101. The composition according to claim 78, wherein the at least one compound is chosen from triethyl acetylcitrate, triethylhexyl acetylcitrate, trihexyl acetylcitrate, trihexyl butyroylcitrate, isodecyl citrate, isopropyl citrate, tributyl citrate and triethylhexyl citrate.

102. The composition according to claim 78, wherein the at least one compound is not tributyl acetylcitrate.

103. The composition according to claim 78, wherein the at least one compound is chosen from butyl benzyl phthalate, dibutyl phthalate, diethylhexyl phthalate, diethyl phthalate and dimethyl phthalate.

104. The composition according to claim 78, wherein the at least one compound is chosen from dibutyl adipate and di-2-ethylhexyl adipate.

105. The composition according to claim 78, wherein the at least one compound is not diisopropyl adipate.

106. The composition according to claim 78, wherein the at least one compound is chosen from dibutyl sebacate, diethylhexyl sebacate, diethyl sebacate and diisopropyl sebacate.

107. The composition according to one of claims 78, wherein the at least one compound is chosen from diethylhexyl succinate and diethyl succinate.

108. A method of cosmetic care or makeup of the lips or skin, comprising applying to the lips or skin a cosmetic composition comprising
a) at least one liquid fatty phase,
b) a dispersion of at least one polymer particle dispersed in the at least one liquid fatty phase, and
c) at least one compound chosen from esters of aliphatic and aromatic polycarboxylic acids and aliphatic and aromatic alcohols comprising from 1 to 10 carbon atoms, the at least one compound being present in an amount sufficient to plasticize the polymer.

109. A method of limiting the transfer and/or enhancing the staying power and/or facilitating the application of a composition for making up or caring for the skin or lips, comprising applying to the skin or lips a composition, wherein the composition comprises
a) at least one liquid fatty phase,
b) a dispersion of at least one polymer particle dispersed in the at least one liquid fatty phase, and
c) at least one compound chosen from esters of aliphatic and aromatic polycarboxylic acids and aliphatic and aromatic alcohols comprising from 1 to 10 carbon atoms, the at least one compound being present in an amount sufficient to plasticize the at least one polymer particle, and introducing into the at least one liquid fatty phase at least one compound having a solubility parameter, δₕ, ranging from 5.5 to 11 (J/cm³)^{1/2}.

110. A method of limiting the transfer and/or enhancing the staying power and/or facilitating the application of a composition for making up or caring for the skin or lips, comprising applying to the skin or lips a composition to the skin or lips, wherein the composition comprises
a) at least one liquid fatty phase,
b) a dispersion of at least one polymer particle dispersed in the at least one liquid fatty phase, and
c) at least one compound chosen from esters of aliphatic and aromatic polycarboxylic acids and aliphatic and aromatic alcohols comprising from 1 to 10 carbon atoms, the at least one compound being present in an amount sufficient to plasticize the at least one polymer particle.

111. A method of makinga composition for application to the skin, lips and epidermal derivatives comprising
adding to the composition particles of at least one polymer dispersed in at least one liquid fatty phase and surface-stabilized by at least one stabilizer,
and at least one compound having a solubility parameter δₕ ranging from 5.5 to 11 (J/cm³)^{1/2}, the at least one compound being present in an amount sufficient to plasticize the at least one polymer particle.

112. A method of making a composition for application to the skin, lips and epidermal integument comprising adding in the composition particles of at least one polymer dispersed in at least one liquid fatty phase and surface-stabilized by at least one stabilizer, and at least one compound chosen from esters of aliphatic and aromatic polycarboxylic acids and aliphatic and aromatic alcohols comprising from 1 to 10 carbon atoms, the at least one compound being present in an amount sufficient to plasticize the at least one polymer particle, to limit the transfer and/or enhance the staying power of the composition over time, and/or to facilitate its application.

113. A cosmetic makeup product comprising
a first composition comprising
a) at least one liquid fatty phase and
b) a dispersion of polymer particles dispersed in the at least one fatty phase, and
c) at least one compound chosen from esters of aliphatic and aromatic polycarboxylic acids and aliphatic and aromatic alcohols comprising from 1 to 10 carbon atoms, the compound being present in an amount sufficient to plasticize the at least one polymer; and
a second composition comprising a physiologically acceptable medium.

114. A cosmetic makeup composition comprising
a first composition comprising
a) at least one liquid fatty phase,
b) a dispersion of polymer particles dispersed in the at least one liquid fatty phase, and
c) at least one compound having a solubility parameter, δₕ, ranging from 5.5 to 11 (J/cm³)^{1/2}, the compound being present in an amount sufficient to plasticize the at least one polymer particle; and
a second composition comprising a physiologically acceptable medium.
